(19) United States
(12) Patent Application Publication
(10) Pub. No.: US 2004/0267365 A1
(43) Pub. Date: Dec. 30, 2004

(54) Device for intersomatic stabilization using a mini-invasive approach

(76) Inventor: Maurizio Fornari, Milano (IT)

Correspondence Address: MERCHANT & GOULD PC, P.O. BOX 2903, MINNEAPOLIS, MN, 55402-0903, US

(21) Appl. No.: 10/484,679

(22) PCT Filed: Jul. 24, 2002
(86) PCT No.: PCT/EP02/08238

(30) Foreign Application Priority Data
Jul. 24, 2001 (IT) TO2001 A 000723

(51) Int. Cl. A61F 2/44

(52) U.S. Cl. 623/17.16; 623/17.11

(57) ABSTRACT

A device (1) for intersomatic stabilization using a mini-invasive approach, which is designed to be inserted between two contiguous vertebrae to keep them at a distance from one another and has a substantially cylindrical body (2) extending along a longitudinal axis (A), and a thread (5) external to and fixed to the body (2) itself; a head end (4) having a cross section that increases along the axis (A) is provided at one end of the body (2) for dilating the intervertebral space as advance of the end (4) itself in the intervertebral space proceeds until there is created with the thread (5) a seat for the subsequent advance of the device (1) having stabilizing function.

The present invention relates to a device for intersomatic stabilization using a mini-invasive approach designed to be inserted between two contiguous vertebrae for keeping the two vertebrae at a distance apart from one another and for favouring their intersomatic fusion.

In degenerative discopathies and other similar conditions that lead to the collapse of an intervertebral disc with a consequent vertebral instability and co-presence of backache, there is known the use of devices for intersomatic stabilization designed to reconstitute the intervertebral space and to restore, simultaneously, spinal stability, which is fully obtained following upon completion of a valid bone fusion between the two vertebrae concerned.

Known devices for intersomatic stabilization, which are generally referred to as “cages”, may be of the screwed type or else of the impact type and entail the use of further tools for the preparation of a threaded seat for the cage and for maintaining the correct intervertebral distance during insertion of the device itself, at the end of which preparatory step a certain amount of homologous or autologous bone is normally inserted for the purpose of facilitating intersomatic fusion.

Once fusion is achieved, the two contiguous vertebrae are perfectly stable, and the implanted cage is completely integrated with attainment of spinal stability, as well as disappearance of pain.

Known surgical techniques resort to different modes of access for the implantation of the above-mentioned devices for intersomatic stabilization. Various modes of access are in fact practicable: posterior, lateral or anterior. These are chosen according to the particular condition to be treated and the inclinations and modes of operating of the surgeon.

Devices for intersomatic stabilization of the type described above and the possibility afforded by them for being inserted only through the customary routes of access pose a number of problems, which are due also to the dimensions of the devices normally commercially available.

In fact, the above devices may cause a significant destruction of the bone stock, both at the level of the compact bone of the discs and at the level of the laminae and of the articular surfaces. Added to this is the further negative aspect that the two contiguous vertebral bodies, which are disadvantageously damaged in their integrity, both anterior and posterior, on account of the intervention of preparation and insertion of the device, also lose their intrinsic stability.

The purpose of the present invention is to provide a device for intersomatic stabilization using a mini-invasive approach, which presents structural and functional characteristics such that the implantation proves significantly less invasive and, especially, such as to guarantee the almost absolute integrity of laminae and articular processes, thus solving the problems described above.

According to the present invention, there is provided a device for intersomatic stabilization using a mini-invasive approach, which is designed to be inserted between two contiguous vertebrae to keep them at a distance from one another and which comprises a substantially cylindrical body extending along a longitudinal axis and a thread external to and fixed to the body itself. The said device is characterized in that it comprises a head end, which is fixed to the body and defined by a solid of revolution of cross section that increases along the axis, the end being designed to dilate the intervertebral space as advance of the end itself proceeds' in the intervertebral space until there is created by the thread a seat for the subsequent advance of the device with stabilizing function.

The invention will now be described with reference to the attached drawings, which illustrate a non-limiting example of embodiment thereof and in which:

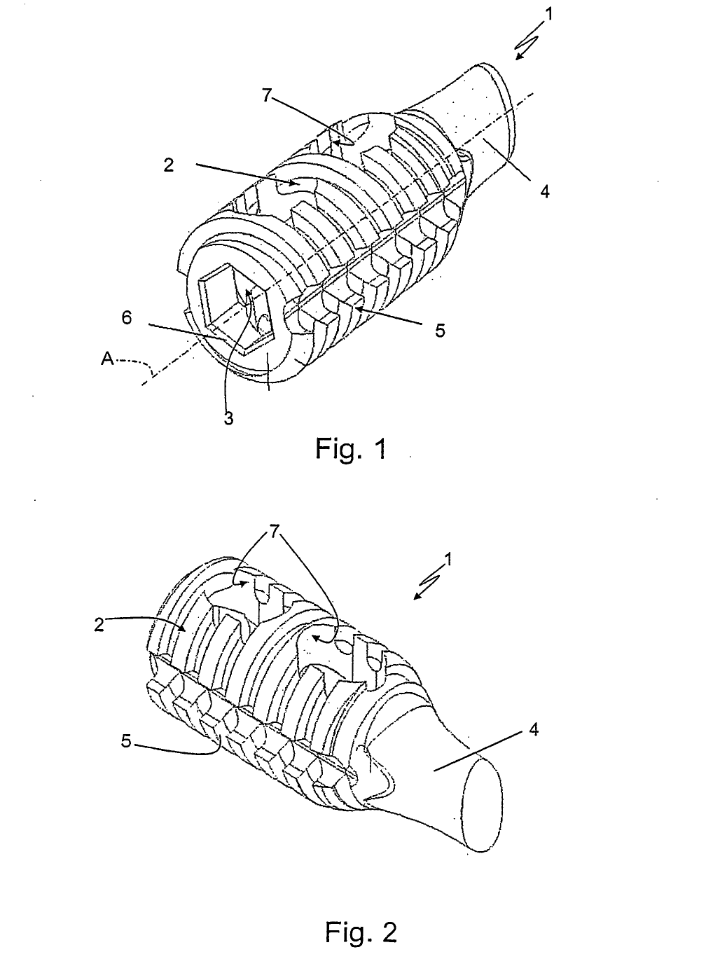

FIG. 1 is a rear perspective view of a first preferred embodiment of the device for intersomatic stabilization using a mini-invasive approach according to the present invention;

FIG. 2 is a front perspective view of the device of FIG. 1;

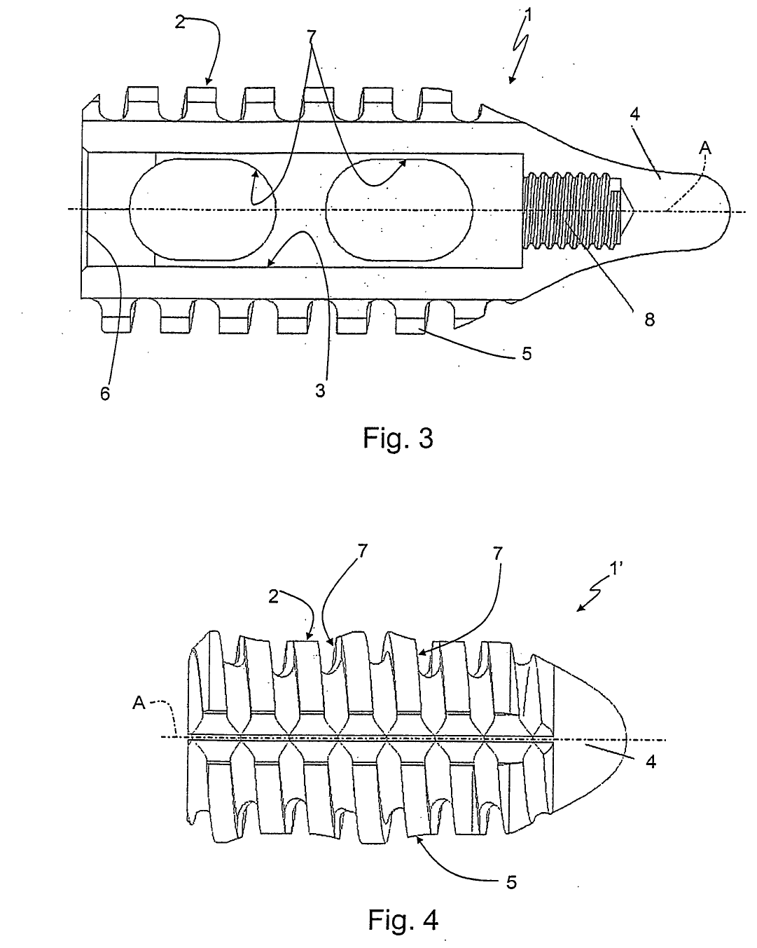

FIG. 3 is a cross-sectional view of the device of FIG. 1 taken according to an axial plane of the device itself; and

FIG. 4 illustrates, in side elevation, a second preferred embodiment of the device of FIG. 1.

With reference to FIGS. 1, 2 and 3, the reference number 1 designates as a whole a device for intersomatic stabilization using a mini-invasive approach designed to be inserted between two contiguous vertebrae for keeping the two vertebrae themselves at a distance apart from one another and for favouring their subsequent intersomatic fusion.

The device 1 comprises a hollow cylindrical body 2, which extends along a respective major longitudinal axis A and is provided with an internal through cavity 3, and a shaped head 4 set at one end of the body 2. The body 2 is moreover provided with a threaded external surface 5, the thread possibly having even more than one start, which extends throughout the body 2 itself starting from the head 4 and enables screwing of the device 1 in the space between the two vertebrae referred to above.

The cavity 3 presents, in a cross section transverse to the axis A, a substantially cylindrical shape, and is provided with a slot 6 with a hexagonal cross section defining a seat for transmission of the tightening torque to the device 1 by a manoeuvring tool, as well as with a further seat 8 set along the axis A and inside the head 4, the said seat 8 being designed to cause the device 1 and the above-mentioned manoeuvring tool to be integrally fixed together.

The cavity 3 is moreover provided with four through holes 7, which are made through the surface to set the cavity 3 in communication with the outside world and are aligned in twos along the axis A and aligned in twos in a direction transverse to the axis A.

The surface 5 has an external diameter that is practically constant along the entire axis A, except for the area immediately adjacent to the slot 6, and the area immediately contiguous to the head 4, which, instead, is tapered towards the head 4 itself and is made in such a way as to be self-tapping, according to technologies that are already known and applied in mechanical engineering.

The head 4 has the task, during insertion of the device 1 between the aforementioned two vertebrae, of appropriately dilating the intervertebral space so as to enable the subsequent screwing of the device 1 itself, and is defined by a solid of revolution of increasing section having a substantially elliptical cross section and a thickness S, which varies as the angular position of the device 1 about the axis A varies.

The head 4 is made so as to enable its insertion in an intervertebral space of limited height and then, by means of a rotation through 90 degrees, to increase the intervertebral space itself up to the height necessary for the surface 5 to be able to start gripping in the bone of the aforementioned two vertebrae, creating in the bone itself a seat for the subsequent advance of the device 1 having stabilizing function.

The device 1 described above renders unnecessary both the dilators used before the introduction of all the cages mentioned above in the introduction and the positioning and maintenance in situ of dilators or spacers that guarantee maintenance of the intersomatic distance in the step of milling and threading of the vertebral bodies prior to introduction of the threaded cages.

The resultant of these two advantages is the availability of a cage having a dilating head 4, which guarantees, during the step of tapping, a correct spontaneous positioning of the head 4 itself between the two vertebral bodies, the said relative distance being maintained as the advance of the head 4 of the body 2 proceeds.

In other words, the device 1 can enable provision of an intersomatic arthrodesis with posterior or monolateral intraforaminal bilateral mini-invasive approach, and, finally, positioning by posterior or posterolateral (intraforaminal) route of the device 1 will not cause any destruction or destabilization of the posterior compartment, providing, instead, an extremely valid intersomatic arthrodesis.

The embodiment illustrated in FIG. 4 relates to a device 1′ similar to the device 1, from which the device 1′ differs in the respect that the head 4 does not have a substantially elliptical cross section, but an ogival shape, and the progressive distancing of the two aforementioned vertebrae is consequently obtained by impacting along the axis A of the body 2.

The device 1′ has an action of impact, unlike the device 1 which, instead, has a lever-type dilating action, nevertheless affording all the advantages provided by the device 1.

It is to be understood that the invention is not limited to the embodiments described and illustrated herein, which are to be considered as examples of embodiment of the device for intersomatic stabilization using a mini-invasive approach, the said device being liable to further modifications as regards the shapes and arrangement of parts, and as regards details of construction and assembly.

1. A device (1) for intersomatic stabilization using a mini-invasive approach designed to be inserted between two contiguous vertebrae to keep them at a distance from one another, and comprising:
a substantially cylindrical body (2), which extends along a longitudinal axis (A);
a thread (5) external to and fixed to the body (2) itself; and
a head end (4), which is fixed to the body (2) and has a cross section that increases along the axis (A);
the device (1) wherein said head end (4) has an elliptical cross section so as to present a thickness (S), which varies as an angular position of the body (2) about the said axis (A) varies, the head end (4) being designed to dilate the intervertebral space as advance of the end (4) itself proceeds in the intervertebral space until there is created with the thread (5) a seat for the subsequent advance of the device (1) having stabilizing function.

2. The device according to claim 1, wherein said head end (4) has an increasing oval cross section so as to dilate the intervertebral space as its advance in the intervertebral space proceeds.

3. The device according to claim 1, wherein said body (2) is provided with seat (6) with a hexagonal cross section for angular coupling of the device (1) itself with a manoeuvring tool.

4. The device according to claim 3, wherein said body (2) comprises four through holes (7), which are made through said thread (5) to set an internal cavity (3) of the body (2) in communication with the outside world, and which are aligned in twos along the axis (A) and aligned in twos in a direction transverse to the axis (A) itself.
